(19) United States
(12) Patent Application Publication
(10) Pub. No.: US 2001/0008940 A1
(43) Pub. Date: Jul. 19, 2001

(54) Novel process

(75) Inventors: Andrew Simon Craig, Sevenoaks (GB); Victor Witold Jacewicz, Tunbridge Wells (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(73) Assignee: SmithKline Beecham plc

(21) Appl. No.: 09/759,513

(22) Filed: Jan. 12, 2001

Related U.S. Application Data
(63) Continuation of application No. 09/179,714, filed on Oct. 27, 1998.

(30) Foreign Application Priority Data
Oct. 27, 1997 (GB) 9722694.8

(51) Int. Cl. A61K 31/4525; C07D 211/00

(52) U.S. Cl. 546/197; 514/321

(57) ABSTRACT

Non-crystalline paroxetine hydrochloride is prepared by precipitation of the same as a solid from a solution of paroxetine hydrochloride, or by drying an oil containing paroxetine hydrochloride, or by removing water/solvent from a hydrate/solvate. The oil may also be obtained by precipitation from a solution of paroxetine hydrochloride.

The present invention relates to a process for the preparation of a pharmaceutically active compound, and to use of the so-prepared compound in therapy. In particular this invention is concerned with the preparation of an non-crystalline form of paroxetine hydrochloride.

Pharmaceutical products with antidepressant and anti-Parkinson properties are described in U.S. Pat. No. 3,912,743 and U.S. Pat. No. 4,007,196. An especially important compound among those disclosed is paroxetine, the (−) trans isomer of 4-(4′-fluorophenyl)-3-(3′,4′-methylene-dioxyphenoxymethyl)-piperidine. This compound is used in therapy as the hydrochloride salt to treat inter alia depression, obsessive compulsive disorder (OCD) and panic.

Paroxetine hydrochloride has been described in the literature as a crystalline hemihydrate (see EP-A-0223403 of Beecham Group) and as various crystalline anhydrate forms (see WO96/24595 of SmithKline Beecham plc).

This invention provides various techniques for preparing non-crystalline forms of paroxetine hydrochloride.

As the first aspect of the present invention, there is provided a method for preparing non-crystalline paroxetine hydrochloride by precipitation of the same from a solution of paroxetine hydrochloride.

Non-crystalline paroxetine hydrochloride may be precipitated as a solid directly from a solution, typically by addition of a suitable preciptitant.

However under some conditions paroxetine hydrochloride may be precipitated from solution as an oil, which on vacuum drying also yields solid non-crystalline paroxetine hydrochloride.

Accordingly, as the second aspect of the present invention, there is provided a method for preparing non-crystalline paroxetine hydrochloride by drying an oil containing paroxetine hydrochloride.

Oils yielding non-crystalline paroxetine hydrochloride may also be prepared by alternative routes as described below.

In a third aspect, the present invention provides a method for preparing non-crystalline paroxetine hydrochloride which comprises removing water or a solvent from a hydrate or solvate of paroxetine hydrochloride, but excluding removal of water from paroxetine hydrochloride hemihydrate and removal of toluene from the toluene solvate of paroxetine hydrochloride.

Paroxetine hydrochloride forms solvates with a wide range of solvents by crystallisation from a solution in the solvent. The solvent may be removed by drying in a heated oven.

The first aspect of this invention preferably involves direct precipitation of a solid from solution. For example, addition of a solution of hydrogen chloride in dry diethyl ether to paroxetine free base in dry diethyl ether results in a crisp solid precipitate which may be collected and vacuum dried providing it is kept dry. Excess water or the presence of some other solvents results in precipitation of an oil instead, which may be processed by the second aspect of the invention.

As an alternative to the addition of a precipitant, a concentrated solution of paroxetine hydrochloride (e.g. in dichloromethane) may be added dropwise to briskly stirred solvent (e.g. n-hexane or diethylether) cooled to, for example, −70° C. At room temperature this procedure would be expected to result in precipitation of an oil except that, as a result of the low temperature, a glass is formed instead. Any solvent trapped in the glass is leached out by the miscible solvent.

In addition to precipitation from a solution as described above, oils may be prepared by a variety of other methods. Oils generally consist of a phase containing paroxetine hydrochloride and one or more solvents, frequently water. These oils crystallise only very slowly in the presence of seeds (probably as a result of the high viscosity) but when subject to drying, especially vacuum drying, are converted into stable non-crystalline solids.

Paroxetine hydrochloride may be dissolved at reflux in a wide range of solvents, e.g. water, ethanol, toluene, methyl isobutyl ketone, to give highly concentrated solutions. For example solutions containing in excess of 30% paroxetine hydrochloride may be prepared in hot water. When such solutions are cooled in the absence of seeds of crystalline forms, an oily phase separates out.

Hygroscopic solid forms of paroxetine hydrochloride may absorb sufficient water to form an oil at ambient temperatures, and other forms of paroxetine hydrochloride may similarly absorb small quantities of other solvents to form oils.

Oils may also be prepared by adding a second solvent to a solution of paroxetine hydrochloride so that the solubility of the paroxetine hydrochloride is exceeded and, in the absence of seeds, causing paroxetine hydrochloride to separate from the solution as an oil.

Hydrogen chloride as a gas or in solution may be added to a solution of paroxetine free base, or to a salt other than the hydrochloride in a suitable solvent, to precipitate paroxetine hydrochloride as an oil.

Some impurities may inhibit crystallisation and promote the formation of oils and non-crystalline precipitates. One example is the common impurity 5,5′-(methylene)bis[6-((4-(4-fluorophenyl)-3-piperidinyl)-methoxy)-1,3-benzodioxole].

Oils obtained by any of these methods may be dried to produce non-crystalline solids.

The non-crystalline product of this invention may be formulated for therapy in the dosage forms described in EP-A-0223403 or WO96/24595, either as solid formulations or for the preparation of solutions for parenteral use.

Therapeutic uses of the paroxetine product of this invention include treatment of: alcoholism, anxiety, depression, obsessive compulsive disorder, panic disorder, chronic pain, obesity, senile dementia, migraine, bulimia, anorexia, social phobia, pre-menstrual syndrome (PMS), adolescent depression, trichotillomania, dysthymia, and substance abuse, referred to below as “the disorders”.

Accordingly, the present invention also provides:

a pharmaceutical composition for treatment or prophylaxis of the disorders comprising solid paroxetine hydrochloride obtainable by the process of this invention and a pharmaceutically acceptable carrier, or a solution of the obtainable solid paroxetine hydrochloride;

the use of solid paroxetine hydrochloride obtainable by the process of this invention to manufacture a medicament in solid or liquid form for the treatment or prophylaxis of the disorders; and

a method of treating the disorders which comprises administering an effective or prophylactic amount of solid paroxetine hydrochloride obtainable by the process of this invention, or a solution thereof, to a person suffering from one or more of the disorders.

The invention is illustrated by the following Examples:

### EXAMPLE 1

(−) trans 4-(4′-fluorophenyl)-3-(3′,4′-methylenedioxyphenoxymethyl)-N-phenoxycarbonyl piperidine (110.9 g) was refluxed for 4 hours with potassium hydroxide (61.7 g) in 2-methoxyethanol (370 ml). The mixture was evaporated to dryness, treated with water (200 ml), extracted three times with benzene (3×200 ml), and the extracts combined and dried with anhydrous magnesium sulphate. The extracts were evaporated to an oil, which was dissolved in ethanol (500 ml) and treated with concentrated hydrochloric acid (27 ml). The ethanol solution was evaporated to a glassy solid and dried over fresh phosphoric oxide at high vacuum to give a crisp free-flowing white solid (96 g).

### EXAMPLE 2

(−) trans 4-(4′-fluorophenyl)-3-(3′,4′-methylenedioxyphenoxymethyl)-piperidine (0.5 g) was dissolved in dry diethyl ether (50 ml). A solution of hydrogen chloride gas (100 mg) in diethyl ether (1.25 ml) was added slowly with stirring. A white solid precipitate resulted which was filtered and dried under high vacuum to give white glassy solid. The infra-red spectrum showed broad bands typical of non-crystalline paroxetine hydrochloride.

### EXAMPLE 3

(−) trans 4-(4′-fluorophenyl)-3-(3′,4′-methylenedioxyphenoxymethyl)-N-phenoxycarbonyl piperidine (2.15 g) was refluxed for 4 hours with potassium hydroxide (1.25 g) in 2-methoxyethanol (7.5 ml). The mixture was evaporated to dryness, treated with water (20 ml), extracted three times with benzene (3×20 ml), and the extracts combined and dried with anhydrous potassium carbonate. The extracts were evaporated to a pale oil, which was treated with a solution of 2 equivalents of hydrogen chloride in diethyl ether (5 ml). An oil separated out from the ether phase and was decanted and dried under high vacuum. The resulting glass was dissolved in ethanol (5 ml) and diluted with diethyl ether (150 ml). Once again, an oil separated out and was decanted and dried under high vacuum to produce a crisp glassy solid.

What is claimed is:

1. A process for preparing non-crystalline paroxetine hydrochloride comprising precipitating the same from a solution of paroxetine hydrochloride.

2. A process according to claim 1 in which the non-crystalline paroxetine hydrochloride is precipitated by addition of a suitable preciptitant.

3. A process according to claim 2 comprising adding of a solution of hydrogen chloride in dry diethylether to a solution of paroxetine free base in dry diethylether.

4. A process for preparing non-crystalline paroxetine hydrochloride comprising drying an oil containing paroxetine hydrochloride.

5. A process according to claim 4 in which the oil containing paroxetine hydrochloride is precipitated a solution of paroxetine hydrochloride.

6. A process according to claim 5 in which the solution contains excess water or another solvent.

7. A process according to claim 5 in which the solution is supersaturated.

8. A process for preparing non-crystalline paroxetine hydrochloride which comprises removing water or a solvent from a hydrate or solvate of paroxetine hydrochloride, but not including removal of water from paroxetine hydrochloride hemihydrate and removal of toluene from the toluene solvate of paroxetine hydrochloride.

9. A pharmaceutical composition for treatment or prophylaxis of the disorders comprising solid paroxetine hydrochloride obtainable by the process of claim 1 and a pharmaceutically acceptable carrier, or a solution of the obtainable solid paroxetine hydrochloride.

10. The use of solid paroxetine hydrochloride obtainable by the process of claim 1 to manufacture a medicament in solid or liquid form for the treatment or prophylaxis of the disorders.

11. A method of treating the disorders which comprises administering an effective or prophylactic amount of solid paroxetine hydrochloride obtainable by the process of claim 1 or a solution thereof, to a person suffering from one or more of the disorders.
